(19) United States
(12) Patent Application Publication
(10) Pub. No.: US 2011/0039467 A1
(43) Pub. Date: Feb. 17, 2011

(54) Ionic liquid flame retardants

(75) Inventor: Yanjie Xu, Longmont, CO (US)

Correspondence Address: Xanjie Xu, 4210 Redmond Dr., Longmont, CO, 80503, US

(73) Assignee: H&C Chemical, Longmont, CO (US)

(21) Appl. No.: 12/806,267

(22) Filed: Aug. 9, 2010

Related U.S. Application Data
(60) Provisional application No. 61/274,031, filed on Aug. 11, 2009.

(51) Int. Cl. B32B 5/02; C07D 233/54; C07D 245/02; C07C 211/64; C07F 9/06; C07C 53/00; C07D 213/20; C09K 21/12; C09K 21/10; C08K 5/52; C08K 5/3417; C08K 5/3432; C08K 5/34; D06M 13/244; D06M 13/352; D06M 13/355; D21H 25/00

(52) U.S. Cl. 442/141; 548/341.1; 540/470; 564/289; 558/207; 562/512; 546/347; 252/601; 524/145; 524/99; 524/106; 524/154; 524/98; 524/186; 8/94.1 R; 8/94.1 P; 162/159; 442/147; 442/146

(57) ABSTRACT

The present invention relates to the use of ionic liquids as flame retardants. The compounds of the invention may be used as flame retardants in various materials without causing damage to the environment and or health of humans or animals. Ionic liquid flame retardants maybe applied alone or in combination with traditional flame retardants. Ionic liquid flame retardants can be applied to finish textile, plastic, leather, paper, rubber, or as wild fire flame retardants.

### CROSS REFERENCE TO RELATED APPLICATIONS

This application claims the benefit of U.S. Provisional Application No. 61/274,031 filed on 11 Aug. 2009 which is incorporated herein in its entirety.

### FIELD OF THE INVENTION

The present invention is broadly directed to novel flame or fire retardant compositions including ionic liquids.

### BACKGROUND OF THE INVENTION

Flame retardants are chemical additives which may be used across a variety of consumer products, such as plastics, textiles, leather, paper, rubber, etc. Chemicals which may be used as flame retardants can be mineral, halogen containing, nitrogen containing and phosphorus containing chemicals, silicon based chemicals, etc. The term “retardant” represents a class of use and not a class of chemical structure.

Preventive flame protection, including the use of flame retardants, has been practiced since ancient times. For example, alum was used to reduce the flammability by Egyptians at the time of about 540 BC. The advent of synthetic polymers earlier last century was of special significance, since the water soluble inorganic salts used up to that time were of little or no utility in these largely hydrophobic materials. Modern developments were, therefore, concentrated on the development of polymer compatible flame retardants. Wild forest fires comprise a serious problem, burning thousands of hectares all over the world each year. Diammonium phosphate (DAP), monoammonium phosphate (MAP), ammonium polyphosphate (APP) and ammonium sulphate (AS) have been used as long-term flame retardants. They are regarded as long-term flame retardants, because they can inhibit combustion even after the loss of their water matrix.

Fundamentally, four processes are involved in polymer flammability: preheating, decomposition, ignition and combustion and propagation. Flame retardants interfere with combustion during a particular stage of this process, i.e. during heating, decomposition, ignition or flame spread through physical or chemical actions.

There are several ways in which the combustion process can be retarded by physical action: for example cooling, formation of a protective layer/coating and/or dilution. During cooling action endothermic processes triggered by flame retardants may cool the material to a temperature below that required to sustain the combustion process. By formation of a protective layer/coating, a condensed combustible layer may be shielded from the gaseous phase with a solid or gaseous protective layer. A condensed phase is thus cooled, smaller quantities of pyrolysis gases are evolved, the oxygen necessary for the combustion process is excluded and heat transfer is impeded. By dilution, the incorporation of inert substances (e.g., fillers) and additives that evolve inert gases on decomposition may dilute the fuel in the solid and gaseous phases so that the lower ignition limit of the gas mixture is not exceeded.

Flame retardants may impede combustion by providing chemical reactions which interfere with combustion processes occurring in the solid and/or gas phases. For reactions in the gas phase, a free radical mechanism of a combustion process which takes place in the gas phase is interrupted by a flame retardant. Exothermic processes may thus be stopped, the system cools down, and the supply of flammable gases is reduced and eventually completely suppressed. For reactions in the solid phase, two types of reaction may take place. Firstly, breakdown of a polymer may be accelerated by a flame retardant, causing pronounced flow of a polymer and, hence, its withdrawal from the sphere of influence of the flame, which breaks away. Secondly, a flame retardant may cause a layer of carbon to form on a polymer surface. This can occur, for example, through the dehydrating action of the flame retardant generating double bonds in the polymer. These may form a carbonaceous layer by cyclizing and cross-linking.

In recent years, there are growing concerns about the safety of these flame retardant chemicals. An issue with the above mentioned forest flame retarding chemicals are their impact on the environment. Initially it was thought that these flame retardants would have no adverse on the environment, as their main active ingredients are agricultural fertilizers. However, ammonia, coming from the dissociation of the ammonium salts, is regarded extremely toxic. Ecotoxicological studies were performed to understand the effects of long-term forest fire retardants on enzymatic activities, cells and microorganisms, thereby obtaining LC50 levels (lethal concentration). The LC50 value of ammonia is 0.53-4.94, which is extremely toxic. Toxicity studies on aquatic organisms relate the results obtained to the determined amount of flame retardants and ammonia. The data show that ammonia is the component that has most impact on these organisms under the testing conditions.

Brominated flame retardants, such as polybrominated diphenylethers (PBDEs), were first introduced into the consumer marketplace in the 1970s. They showed great compatibility with plastics and textiles, and offered superior flame retardant properties. Brominated flame retardants interrupt combustion by volatizing bromine radicals to react with high energetic free radicals O. and .OH from the combustion, thereby preventing the spread of the flame. The most commonly used brominated flame retardants are PBDEs and tetrabromobisphenol A (TBBPA). By 2010, the brominated flame retardants market is projected to reach 1.7 billion pounds. Market Report by Peter Dufton; 2003

Great efforts are being put into developing halogen free flame retardants, especially phosphorus based flame retardants. However, their flame retarding performance is not satisfactory. The prior art describes the use of some phosphonium ion salts. Doring et al describe polyphosphonium cations with selected anions as flame retardants in application US20100160476. Japanese patent application JP 2010163396 describes straight chain alkylaryl phosphonium salt structures as polymer dopants for high conductivity, heat resistance and flame retardancy. Tan et al have reported fireproofing agent containing quaternary phosphonium salt-modified montmorillonite as flame retardants. A review by Guo in Zhongguo Pige describes development and applications of tetrakis(hydroxymethyl) phosphonium salts as flame retardants among other uses. Ammonium surfactants have been employed to modify the surface of nanoclays for flame retarding application.

Despite health and environmental concerns, the world flame retardant chemicals market is projected to reach 5.7 billion pounds by the year 2012. The United States is the country with the tightest flame safety standards, and consequently the greatest use of brominated flame retardants. Nearly 98 percent of roughly 8,500 metric tons of PBDE used globally is consumed in US. However, brominated flame retardants are not chemically bound to the textiles and many substrates in plastic composites; therefore, they may easily escape into environment. There is growing concern over the persistence and bioaccumulation of brominated flame retardants and their risk to the environment and human health. Since brominated flame retardants are lipophilic and bioaccumulative substances, they may build up in fatty tissues once they enter a human or animal body. Studies have found bromated flame retardants to be widespread in the environment and in human tissues. Studies also have shown that these brominated flame retardants are toxic and can cause serious health disorders. In addition, women in North America have the highest levels globally of these chemicals in their breast milk.

The foregoing examples of the related art and limitations are intended to be illustrative and not exclusive. Other limitations of the related art will become apparent to those of skill in the art upon a reading of the specification and a study of the drawings or figures as provided herein.

### SUMMARY OF THE INVENTION

Therefore a continuing need exists for flame retardant compounds that are environmentally benign and nonmigrating. Ionic liquids show excellent resistance to migration and leaching and do not accumulate in fatty tissue causing toxicity. Additionally, incorporating biodegradable groups can make ionic liquids ready biodegradable and completely non-toxic. The following embodiments, aspects and variations thereof are exemplary and illustrative and not intended to be limiting in scope.

In one aspect, there is provided a method of imparting a flame retarding property to a material comprising treating said material with an effective flame retarding amount of the composition of the formula:

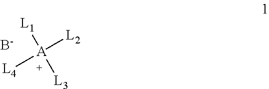

Wherein A is independently selected from a nitrogen, phosphorus or sulfur;

when A is nitrogen L₁, L₂, L₃ and L₄ are each independently selected from R₁, R₂, R₃ and R₄ and wherein R₁, R₂, R₃ and R₄ each independently form a single bond with N in a cyclic or acyclic structure; or, R₁, R₂ and R₃ combine to form an aromatic heterocycle further substituted by R₁, R₂, R₃ and R₄ bonded to N; R₁, R₂, R₃ and R₄ are each independently selected from the group consisting of hydrogen, alkyl, aryl, heterocyclyl, (C₁C₈)cycloalkyl, hetrocyclyl(C₁C₈)alkyl, aryl(C₁C₈)alkyl, heteroaryl and heteroaryl(C₁C₈)alkyl group that may be substituted or unsubstituted by halo, nitro, trifluoromethyl, trifluoromethoxy, methoxy, carboxy, NH₂, OH, SH, NHCH₃, N(CH₃)₂, SMe and cyano; R₁, R₂, R₃ and R₄ are not straight chain unsubstituted alkyl bonded to a quarterany N;
when A is sulfur L₁ and L₂ are R₁₂ and L₃ and L₄ are R₁₃ and R₁₄. R₁₂, R₁₃ and R₁₄ is selected from the group consisting of hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by halo, nitro, trifluoromethyl, trifluoromethoxy, methoxy, carboxy, NH₂OH, SH, NHCH₃, N(CH₃)₂, SMe, cyano and the like;
when A is phosphorus, L1, L2, L3 and L4 are R8, R9, R10 and R11 wherein R8, R9, R10 and R11 are each independently selected from the group consisting of hydrogen, alkyl, aryl, heterocyclyl, (C1C8)cycloalkyl, hetrocyclyl(C1C8)alkyl, aryl(C1C8)alkyl, heteroaryl and heteroaryl(C1C8)alkyl group that may be substituted or unsubstituted by halo, nitro, trifluoromethyl, trifluoromethoxy, methoxy, carboxy, NH2, OH, SH, NHCH3, N(CH3)2, SMe, cyano and the like and wherein R8, R9, R10, R11 is not a hydroxymethyl group; when A is nitrogen and sulfur, B- is X— and X— is selected from the group consisting of [PF6]-[NTf2]-, [BR1R2R3R4]-, [BF4]-, OH—, SCN—, SbF6-, R2PO4, RSO3-, RSO4, OTf-, tris(trifluoromethylsulfonyl)methide, [N(CN)2]-, [CH3CO2]-, [CF3CO2]-, [NO3]-, Br—, Cl—, 1-, [Al2Cl7]-, [AlCl4]-, oxalate, dicarboxylates and tricarboxylate, formate, phosphate and aluminate or a suitably substituted negatively charged functional group on an alkyl, aryl, heterocyclyl, (C1C8)cycloalkyl, hetrocyclyl(C1C8)alkyl, aryl(C1C8)alkyl, heteroaryl or heteroaryl(C1C8)alkyl group that may be substituted or unsubstituted;
when A is phosphorus B⁻ is X₁⁻ and X₁⁻ selected from the group consisting of [PF₆]⁻, [NTf₂]⁻[BR₁R₂R₃R₄]⁻, [BF₄]⁻, OH⁻, SCN⁻, SBF₆⁻, R₂PO₄⁻, RSO₃⁻, RSO₄, OTf⁻, tris(trifluoromethylsulfonyl)methide [N(CN)₂]⁻, [CH₃CO₂]⁻, [CF₃CO₂]⁻, [NO₃]⁻, [Al2Cl₇]⁻, [AlCl₄]⁻, oxalate, dicarboxylates and tricarboxylate, formate, phosphate, I⁻ and aluminate and the like or a suitably substituted negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted and wherein X₁⁻ is not a Br⁻ and Cl⁻; when one of the four R₈, R₉, R₁₀, R₁₁ group is a C₁ to C₁₈ (CH₂)ₙ chain bonded to P., X₁⁻ is not SBF6, PF6, BF4, AlF6, triflate, AsF6, (B[C6F5]4⁻), (B[C6H3(C6H3(CF3)2]4⁻), tetra phenyl borate, hexafluorotitanate, pentachlorotitanate, pentachlorostannate, hexafluorogermanate, hexafluorosilicate, hexafluoronickelate, or hexafluorozirconate

In a variation of the above method, there is provided a flame retardant composition of the formula:

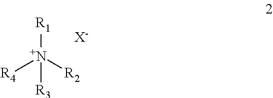

Wherein, R₁, R₂, R₃, R₄ form four bonds with N in a cyclic or acyclic structure; or, R₁, R₂, R₃ combine to form a aromatic heterocycle further substituted by R₁, R₂, R₃ and L₄ is R₄ bonded to N;

R, R₁, R₂, R₃, R₄ refer to an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like;
R, R₁, R₂, R₃, R₄ may a reactive group that serves to bond the ionic liquid into a polymer such as a vinyl, epoxide, acrylate, isocyanate, acyl halide; R₁, R₂, R₃ and R₄ are not straight chain unsubstituted alkyl bonded to a quarterany N; and,
X⁻ is an anion [PF₆]⁻, [NTf₂]⁻, [BR₁R₂R₃R₄]⁻, [BF₄]⁻, OH⁻, SCN⁻, SbF6⁻, R₂PO₄⁻, RSO₃⁻, RSO₄, OTf⁻, tris(trifluoromethylsulfonyl)methide, [N(CN)₂]⁻, [CH₃CO₂]⁻, [CF₃CO₂]⁻, [NO₃]⁻, Br⁻, Cl⁻, I⁻, [Al₂Cl₇]⁻, [AlCl₄]⁻, oxalate, dicarboxylates and tricarboxylate, formate, phosphate, aluminate and the like or a suitably substituted negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted.

In a variation of the above method there is provided a compound of the formula:

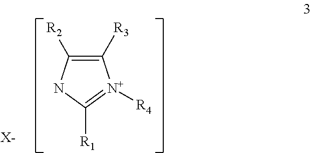

In a variation of the above method there is provided a compound of the formula:

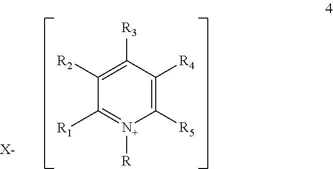

Wherein R and R₅ are an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like; R and R₅ may a reactive group that serves to bond the ionic liquid into a polymer such as a vinyl, epoxide, acrylate, isocyanate, acyl halide

In a variation of the above method there is provided a compound of the formula:

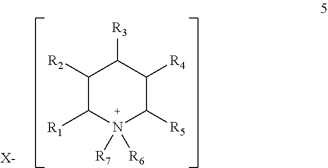

Wherein R₅ R₆, and R₇ are an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like; R₅ R₆, and R₇ may be a reactive group that serves to bond the ionic liquid into a polymer such as a vinyl, epoxide, acrylate, isocyanate, acyl halide

In a variation of the above method there is provided a compound of the formula:

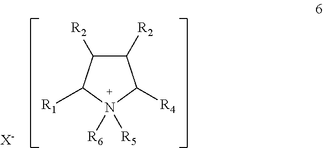

Wherein R₆ and R₅ are an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that maybe substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like;

R₆ and R₅ may a reactive group that serves to bond the ionic liquid into a polymer such as a vinyl, epoxide, acrylate, isocyanate, acyl halide

In a variation of the above method there is provided a compound of the formula:

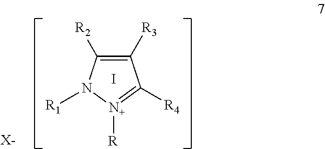

Wherein R is defined in an embodiment above

In a variation of the above method there is provided a compound of the formula:

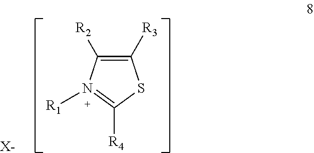

In another aspect, there is provided a method of imparting a flame retarding property to a material comprising treating said material with an effective flame retarding amount of the composition of the formula:

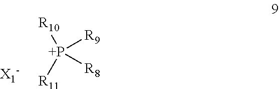

R_{8,9,10,11} refer to an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that maybe substituted or unsubstituted. R_{8,9,10,11} may be a reactive group that serves to bond the ionic liquid into a polymer such as a vinyl, epoxide, acrylate, isocyanate, acyl halide optionally be halo, nitro, trifluoromethyl, trifluoromethoxy, methoxy, carboxy, NH₂, OH, SH, NHCH₃, N(CH₃)₂, SMe, cyano and the like; X₁⁻ selected from the group consisting of [PF₆]⁻, [NTf₂]⁻, [BR₁R₂R₃R₄]⁻, [BF₄]⁻, OH⁻, SCN⁻, SBF₆⁻, R₂PO₄⁻,
RSO₃⁻, RSO₄, OTf⁻, tris(trifluoromethylsulfonyl)methide [N(CN)₂]⁻, [CH₃CO₂]⁻, [CF₃CO₂]⁻, [NO₃]⁻, [Al2Cl₇]⁻, [AlCl₄]⁻, oxalate, dicarboxylates and tricarboxylate, formate, phosphate, I⁻ and aluminate and the like or a suitably substituted negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted and wherein X₁⁻ is not a Br⁻ and Cl⁻; when one of the four R₈, R₉, R₁₀, R₁₁ group is a C₁ to C₁₈ (CH₂)ₙ chain bonded to P., X₁⁻ is not SbF₆, PF₆, BF₄, AlF₆, triflate, AsF₆, (B[C₆F₅]₄⁻), (B[C₆H₃(C₆H₃(CF₃)₂]₄⁻), tetra phenyl borate, hexafluorotitanate, pentachlorotitanate, pentachlorostannate, hexafluorogermanate, hexafluorosilicate, hexafluoronickelate, or hexafluorozirconate. Some other examples are found in FIG. 4.

In another aspect, there is provided a method of imparting a flame retarding property to a material comprising treating said material with an effective flame retarding amount of the composition of the formula:

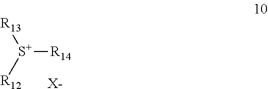

Wherein, R₁₂, R₁₃, R₁₄ refer to an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxy, NH₂, OH, SH, NHCH₃, N(CH₃)₂, SMe, cyano and the like; R₁₂, R₁₃, R₁₄ may a reactive group that serves to bond the ionic liquid into a polymer such as a vinyl, epoxide, acrylate, isocyanate, acyl halide; X⁻ is [PF_{6]-}, [_{NT}f₂]₋, [BR₁R₂R₃R₄]⁻, [BF₄]⁻, OH⁻, SCN⁻, SbF₆⁻, R₂PO₄⁻, RSO₃⁻, RSO₄, OTf⁻, tris(trifluoromethylsulfonyl)methide, [N(C_{N})₂]⁻, [CH₃CO₂]⁻, _{[}CF₃CO₂]⁻, [NO₃]⁻, Br⁻, Cl⁻, I⁻, [Al_{2C}l₇]⁻, [AlCl₄]⁻, oxalate, dicarboxylates and tricarboxylate, formate, phosphate, aluminate and the like or a suitably substituted negatively charged functional group on an alkyl, aryl, heterocyclyl, (C₁C₈)cycloalkyl, hetrocyclyl(C₁C₈)alkyl, aryl(C1C8)alkyl, heteroaryl or heteroaryl(C1C8)alkyl group that may be substituted or unsubstituted. Some other examples are found in FIG. 4

In a variation there is provided a method of imparting a flame retarding property to a material comprising treating said material with an effective flame retarding amount of the composition of the formulas above in combination with other ionic liquid compounds.

In a variation there is provided a flame retardant comprising formula A⁻B⁺ wherein the cationionic or the anionic species is an ionic liquid ion and its counter ion is an ion bonded to a polymer.

In a variation there is provided a method of imparting a flame retarding property to a material comprising treating said material with an effective flame retarding amount of the composition of the formulas above in combination with a mineral flame retardant.

In a variation there is provided a method of imparting a flame retarding property to a material comprising treating said material with an effective flame retarding amount of the composition of the formulas above in combination with a metal hydroxide, hydroxyl carbonate, borates the like.

In another variation there is provided a method of imparting a flame retarding property to a material comprising treating said material with an effective flame retarding amount of the composition of the formulas above combined with a organic flame retardant.

In a variation there is provided a method of imparting a flame retarding property to a material comprising treating said material with an effective flame retarding amount of the composition of the formulas above combined with a halogenated flame retardant.

In a variation there is provided a method of imparting a flame retarding property to a material comprising treating said material with an effective flame retarding amount of the composition of the formulas above combined with halogenated flame retardant additives, halogenated monomers and copolymers which are reactive flame retardants, and the like.

In a variation there is provided a method of imparting a flame retarding property to a material comprising treating said material with an effective flame retarding amount of the composition of the formulas above combined with a phosphorus based flame retardant.

In a variation of the above composition there is provided a flame retardant composition comprising an ionic liquid combined with red phosphorus, inorganic phosphorus, organic phosphorus based compounds, intumescent flame retardant systems and the like.

In a variation there is provided a method of imparting a flame retarding property to a material comprising treating said material with an effective flame retarding amount of the composition of the formulas above combined with a nitrogen based flame retardant,

In a variation there is provided a method of imparting a flame retarding property to a material comprising treating said material with an effective flame retarding amount of the composition of the formulas above combined with silicon based flame retardants.

In a variation there is provided a method of imparting a flame retarding property to a material comprising treating said material with an effective flame retarding amount of the composition of the formulas above combined with silicones, silica and the like

In a variation there is provided a method of imparting a flame retarding property to a material comprising treating said material with an effective flame retarding amount of the composition of the formulas above combined with nanometric particles.

In a variation there is provided a method of imparting a flame retarding property to a material comprising treating said material with an effective flame retarding amount of the composition of the formulas above combined with a nanoclay, carbon nanotubes, nanoscale particulate additives.

In a variation there is provided a method for imparting a flame retarding property to a textile material comprising treating said textile with an effective flame retarding amount of an ionic liquid.

In a variation there is provided a method for imparting a flame retarding property to a plastic material comprising treating said combustable plastic material with an effective flame retarding amount of an ionic liquid.

In a variation there is provided a method for imparting a flame retarding property to a leather comprising treating said leather with an effective flame retarding amount of an ionic liquid.

In a variation there is provided a method for imparting a flame retarding property to paper comprising treating said paper with an effective flame retarding amount of an ionic liquid.

In a variation there is provided a method for imparting a flame retarding property to wood comprising treating said wood with an effective flame retarding amount of an ionic liquid.

In a variation there is provided a method for imparting a flame retarding property to a combustible rubber comprising treating said rubber with an effective flame retarding amount of an ionic liquid.

In a variation there is provided a method for using ionic liquids as wild fire retardant.

In a variation there is provided a plastic composition comprising an ionic liquid flame retardant.

In a variation there is provided a textile composition comprising an ionic liquid flame retardant.

In a variation there is provided a wood composition comprising an ionic liquid flame retardant.

In a variation there is provided a paper composition wood comprising an ionic liquid flame retardant.

In a variation there is provided a leather composition wood comprising an ionic liquid flame retardant.

In a variation there is provided a rubber composition wood comprising an ionic liquid flame retardant.

In a variation there is provided a method of imparting a flame retarding property to a material comprising treating said material with an effective flame retarding amount of the composition of the formulas above also functioning as a dispersant.

In a variation there is provided a method of imparting a flame retarding property to a material comprising treating said material with an effective flame retarding amount of the composition of the formulas above also functioning as a plasticizer.

In a variation there is provided a method of imparting a flame retarding property to a material comprising treating said material with an effective flame retarding amount of the composition of the formulas above also functioning as an antibacterial.

In a variation there is provided a method of imparting a flame retarding property to a material comprising treating said material with an effective flame retarding amount of the composition of the formulas above also functioning as a lubricant.

In a variation there is provided a method of imparting a flame retarding property to a material comprising treating said material with an effective flame retarding amount of the composition of the formulas above also functioning as an anti-corrosion agent.

When reference is made to compounds throughout this disclosure R, R₁, R₂, R₃, R₄, R₅, R₆, R₇, R₈, R₉, R₁₀, R₁₁, R₁₂ R₁₃, R₁₄, refer to an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by be a functional group like halo, nitro, trifluoromethyl, trifluoromethoxy, methoxy, carboxy, NH₂, OH, SH, NHCH₃, N(CH₃)₂, SMe, cyano and the like. R, R₁, R₂, R₃, R₄, R₅, R₆, R₇, R₈, R₉, R₁₀, R₁₁, R₁₂ R₁₃, R₁₄, may a reactive group that serves to bond the ionic liquid into a polymer such as a vinyl, epoxide, acrylate, isocyanate, acyl halide.

When reference is made to compounds throughout this disclosure R₈, R₉, R₁₀, R₁₁ refers to an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by be optionally substituted by halo, nitro, trifluoromethyl, trifluoromethoxy, methoxy, carboxy, NH2, OH, SH, NHCH₃, N(CH₃)₂, SMe, cyano and the like. R₈, R₉, R₁₀, R₁₁ may be a reactive group that serves to bond the ionic liquid into a polymer such as a vinyl, epoxide, acrylate, isocyanate, acyl halide and may. R₈, R₉, R₁₀, R₁₁ is not a hydroxymethyl group,

When reference is made to the negatively charged X⁻ throughout this disclosure X⁻ refers to an anionic species including but not limited to OH⁻, SCN⁻, S_{B}F₆⁻, R₂PO₄⁻, RₛO₃₋, RSO₄⁻, [PF₆]⁻, [NTf₂]⁻, [BR₁R₂R₃R₄]⁻, [BF₄]⁻, OTf⁻, [N(CN)₂]⁻, [CH₃CO₂]⁻, [CF₃CO₂]⁻, [NO₃]₋, Br⁻, Cl⁻, I⁻, [Al2Cl₇]⁻, [AlCl₄]⁻, oxalate, dicarboxylates and tricarboxylate, formate, phosphate, aluminate and the like and a negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted. Some other examples are found in FIG. 4.

When reference is made to the negatively charged X₁⁻ throughout this disclosure X₁⁻ refers to an anionic species including but not limited to OH⁻, SCN⁻, SBF₆⁻, R₂PO₄⁻, RSO₃⁻, RSO₄ [PF₆]⁻, [NTf₂]⁻, [BR₁R₂R₃R₄]⁻, [BF₄]⁻, OTf⁻, [N(CN)₂]⁻, [CH₃CO₂]⁻, [CF₃CO₂]⁻, [NO₃]⁻, [Al2Cl₇]⁻, [AlCl₄]⁻, I⁻, oxalate, dicarboxylates and tricarboxylate, formate, phosphate, aluminate and the like and a negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted. X₁⁻ is not Cl⁻, Br⁻. Some other examples are found in FIG. 4.

The ionic liquid flame retardant compositions of the invention maybe derived from biofeedstock such as carbohydrates, amino acids, fatty acids, nucleotides and other organic and inorganic chemicals derived from biofeedstock.

Some of the compounds of the invention may exist as multi-charged species such as zwitter ions. Certain of the compounds of the present invention can exist in combinations with other compounds and polymers as unsolvated forms as well as solvated forms, including hydrated forms, and are intended to be within the scope of the present invention. Certain of the above compounds may also exist in one or more solid or crystalline phases or polymorphs.

Compounds of this invention, or derivatives thereof, may posses a reactive function such as an alkene, acrylate, isocyanate, acid chloride, epoxide or other functional group that enables bonding to other compounds and polymers and imparts flame retarding properties to said compounds and polymers.

In addition to the exemplary embodiments, aspects and variations described above, further embodiments, aspects and variations will become apparent by reference to the drawings and figures and by examination of the following descriptions.

### DESCRIPTION OF DRAWINGS

FIG. 1 shows structures of heterocyclic and acyclic embodiments of ionic liquids of the invention.

FIG. 2 shows structures of biodegradable ionic liquids.

FIG. 3 shows embodiments of ionic liquids with a reactive group that serves to bond the ionic liquid into a polymer.

FIG. 4 shows embodiments of anionic species used in ionic liquid flame retardants.

### DETAILED DESCRIPTION OF THE INVENTION

### Definitions

Unless specifically noted otherwise herein, the definitions of the terms used are standard definitions used in the chemical arts. Exemplary embodiments, aspects and variations are illustrative in the figures and drawings, and it is intended that the embodiments, aspects and variations, and the figures and drawings disclosed herein are to be considered illustrative and not limiting.

When reference is made to compounds throughout this disclosure R, R₁, R₂, R₃, R₄ refer to an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted. R, R₁, R₂, R₃, R₄ may optionally be halo, nitro, trifluoromethyl, trifluoromethoxy, methoxy, carboxy, NH₂, OH, SH, NHCH₃, N(CH₃)₂, SMe, cyano and the like.

When reference is made to compounds throughout this disclosure R₈, R₉, R₁₀, R₁₁ refers to an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted. R₅ may optionally be halo, nitro, trifluoromethyl, trifluoromethoxy, methoxy, carboxy, NH₂, OH, SH, NHCH₃, N(CH₃)₂, SMe, cyano and the like. R₅ is not a hydroxymethyl group, One of the four R₈, R₉, R₁₀, R₁₁ groups is not a C₁ to C18 (CH₂)ₙ chain bonded to P. R₈, R₉, R₁₀, R₁₁ is not a hydroxymethyl group.

When reference is made to the negatively charged X⁻ throughout this disclosure X⁻ refers to an anionic species like [PF₆]⁻, [NTf₂]⁻, [BR₁R₂R₃R₄]⁻, [BF₄]⁻, OH⁻, SCN⁻, SbF₆⁻, R₂PO₄⁻, RSO₃⁻, RSO₄, OTf⁻, tris(trifluoromethylsulfonyl)methide, [N(CN)₂]⁻, [CH₃CO₂]⁻, [CF₃CO₂]⁻, [NO₃]⁻, Br⁻, Cl⁻, I⁻, [Al₂Cl₇]⁻, [AlCl₄]⁻, oxalate, dicarboxylates and tricarboxylate, formate, phosphate, aluminate and the like or a suitably substituted negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted.

When reference is made to the negatively charged X₁⁻ throughout this disclosure X₁⁻ refers to an anionic species like [PF₆]⁻, [NTf₂]⁻, [BR₁R₂R₃R₄]⁻, [BF₄]⁻, OH⁻, SCN⁻, SBF₆⁻, R₂PO₄⁻, RSO₃⁻, RSO₄, OTf⁻, tris(trifluoromethylsulfonyl)methide [N(CN)₂]⁻, [CH₃CO₂]⁻, [CF₃CO₂]⁻, [NO₃]⁻, [Al2Cl₇]⁻, [AlCl₄]⁻, oxalate, dicarboxylates and tricarboxylate, formate, phosphate, I⁻, aluminate and the like or a suitably substituted negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted. X₁⁻ is not Cl⁻, Br⁻.

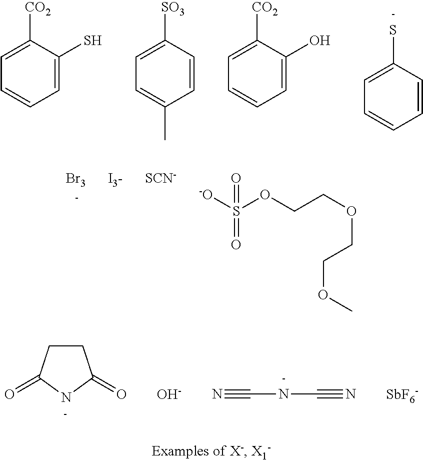

An organic group is an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted.

An “alkyl” group is a straight, branched, saturated or unsaturated, aliphatic group having a chain of carbon atoms, optionally with oxygen, nitrogen or sulfur atoms inserted between the carbon atoms in the chain or as indicated. A (C₁C₂₀)alkyl, for example, includes alkyl groups that have a chain of between 1 and 20 carbon atoms, and include, for example, the groups methyl, ethyl, propyl, isopropyl, vinyl, allyl, 1propenyl, isopropenyl, ethynyl, 1propynyl, 2propynyl, 1,3-butadienyl, penta-1,3-dienyl, penta-1,4-dienyl, hexa-1,3-dienyl, hexa-1,3,5-trienyl, and the like. An alkyl group may also be represented, for example, as a (CR¹R²)ₘ, group where R¹ and R² are independently hydrogen or are independently absent, and for example, m is 1 to 8, and such representation is also intended to cover both saturated and unsaturated alkyl groups.

An alkyl as noted with another group such as an aryl group, represented as “arylalkyl” for example, is intended to be a straight, branched, saturated or unsaturated aliphatic divalent group with the number of atoms indicated in the alkyl group (as in (C₁C₂₀)alkyl, for example) and/or aryl group (as in (C₅C₁₄)aryl, for example) or when no atoms are indicated means a bond between the aryl and the alkyl group. Nonexclusive examples of such group include benzyl, phenethyl and the like.

An “alkylene” group is a straight, branched, saturated or unsaturated aliphatic divalent group with the number of atoms indicated in the alkyl group; for example, a (C₁C₃)alkylene or (C₁C₃)alkylenyl.

A “cyclyl” such as a monocyclyl or polycyclyl group includes monocyclic, or linearly fused, angularly fused or bridged polycycloalkyl, or combinations thereof. Such cyclyl group is intended to include the heterocyclyl analogs. A cyclyl group may be saturated, partically saturated or aromatic.

“Halogen” or “halo” means fluorine, chlorine, bromine or iodine.

A “heterocyclyl” or “heterocycle” is a cycloalkyl wherein one or more of the atoms forming the ring is a heteroatom that is a N, O, or S. Non-exclusive examples of heterocyclyl include piperidyl, 4-morpholyl, 4-piperazinyl, pyrrolidinyl, 1,4-diazaperhydroepinyl, 1,3-dioxanyl, and the like.

Salts include acid addition salts formed with inorganic acids such as hydrochloric acid, hydrobromic acid, sulfuric acid, phosphoric acid, and the like; or with organic acids such as acetic acid, propionic acid, hexanoic acid, malonic acid, succinic acid, malic acid, citric acid, gluconic acid, salicylic acid and the like.

“Substituted or unsubstituted” or “optionally substituted” means that a group such as, for example, alkyl, aryl, heterocyclyl, (C₁C₈)cycloalkyl, hetrocyclyl(C₁C₈)alkyl, aryl(C₁C₈)alkyl, heteroaryl, heteroaryl(C₁C₈)alkyl, and the like, unless specifically noted otherwise, may be unsubstituted or, may substituted by 1, 2 or 3 substitutents selected from the group such as halo, nitro, trifluoromethyl, trifluoromethoxy, methoxy, carboxy, NH₂, OH, SH, NHCH₃, N(CH₃)₂, SMe, cyano and the like.

The present disclosure may be understood by reference to the following detailed description taken in conjunction with the drawings described below.

To replace brominated flame retardants and other chemical compounds that may have toxic bioaccumulative effects; a different class of materials, namely ionic liquids (“IL”), may be used for the purpose of flame retarding.

An ionic liquid is a salt in which the ions are poorly coordinated. At least one ion in the salt has a delocalized charge and one component is organic, which prevents the formation of a stable crystal lattice.

Ionic liquids have capabilities to form a wide range of intermolecular interactions that include strong and weak ionic, hydrogen boding, van der waals, dispersive, pie-pie interactions. Ionic liquids exhibit compatibility with a wide variety of materials including salts, fats, proteins, amino acids, surfactants, oils, inks and plastics, even DNA. Ionic liquids are intensively studied for many applications, such as solvents, catalysts, separation, extraction, biomass processing, etc. ILs have been used as plasticizers, dispersants, and lubricants. When used as plasticizers, they show excellent resistance to migration and leaching which mitigates one of the most significant issues with current flame retardant compounds.

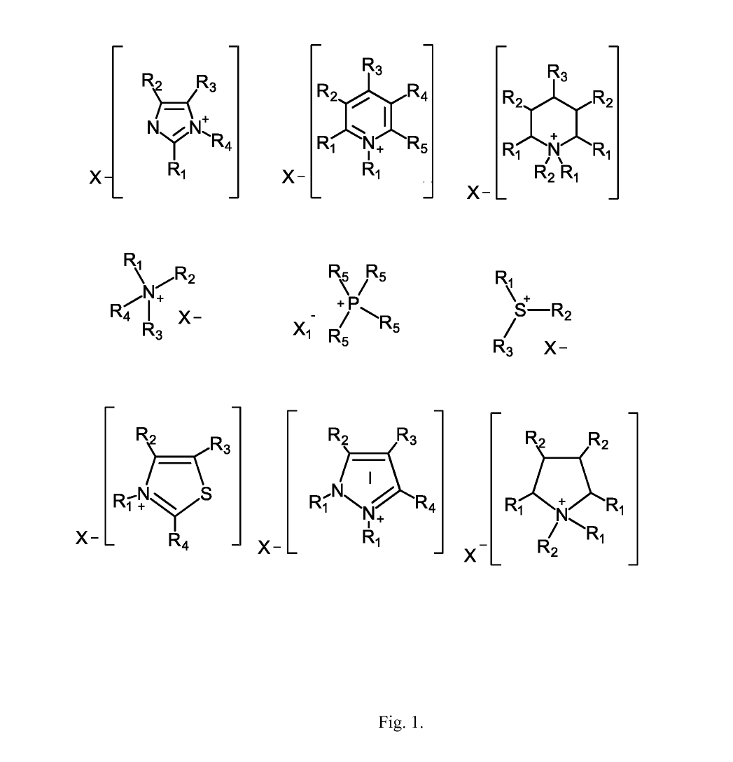

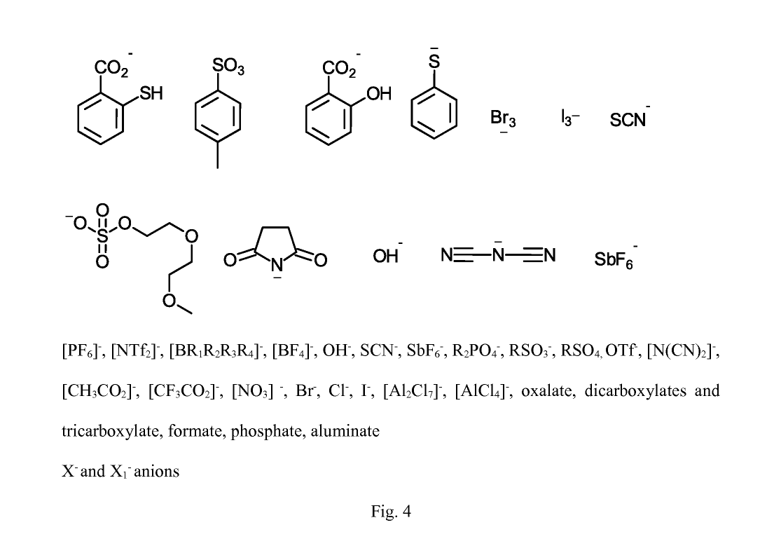

Ionic liquid flame retardants may be suitably configured by selection of cations and anions chosen from, but not limited to, those shown in FIGS. 1, 3 and 4

Ionic liquids are compounds which may contain halogen, nitrogen, phosphate, sulfur, or some combination of these elements. Ionic liquid compounds may be designed with halogen, nitrogen, phosphorus or some combinations of these elements, and so be used solely as flame retardants, either though physical action or chemical action to inhibit combustion processes as discussed above.

Due to the large number of possible combinations of ion pairs, the ability to select the physical and chemical properties of possible ionic liquid flame retardants is essentially unlimited. Functionalization of a ligand or “head”, such as by changing the length of a ligand R group, adding a ligand to different positions of a head, and/or adding a halogen to a ligand or head further increases the number of possible ionic liquid flame retardants. The head may be defined as the positively charged core atom or ring of the cation species of the ionic liquid.

In one embodiment, ionic liquids are modified to design biodegradable and nontoxic ionic liquids via incorporation of ethereal side chains. One such example is shown in FIG. 2. Greener Solvents; Room Temperature Ionic Liquids from Biorenewable Sources, Scott Handy, Chem. Eur. J. 2003, 9, 2938-2944

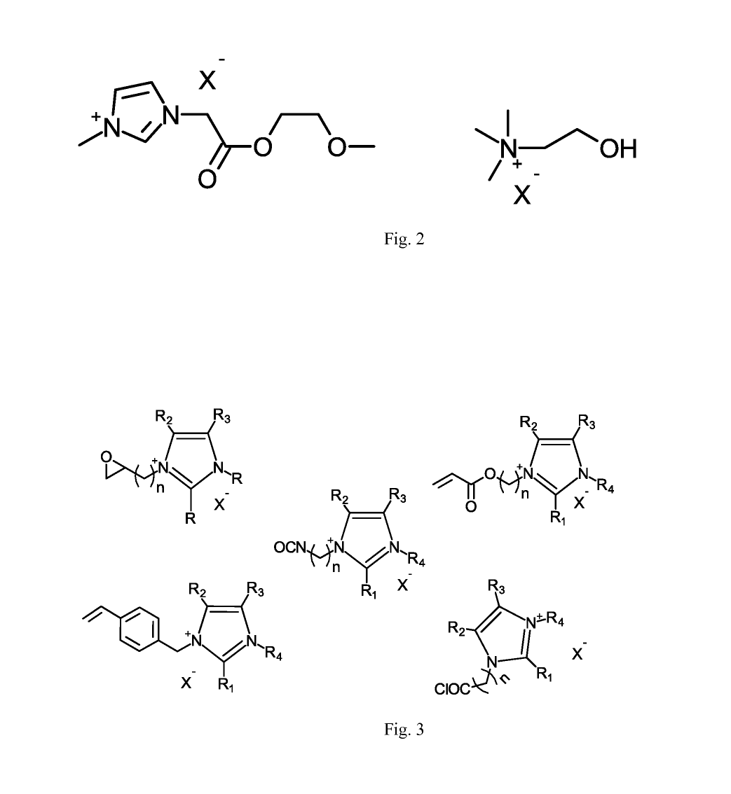

In another embodiment incorporation of reactive groups into ligands, produces ionic liquids which may be chemically bound with a substrate to impart flame retarding properties to substrates. Five such examples are shown in FIG. 3. Other reactive groups may include, but are not limited to hydroxyl and/or carboxyl groups.

In another embodiment, ionic liquids may be formulated with other ionic liquids, or traditional flame retardants or additives. These traditional flame retardants can be mineral flame retardants, halogen containing flame retardants, phosphorous based flame retardants, nitrogen based flame retardants, silicon based flame retardants, nanometric particles, etc. Mineral flame retardants can be metal hydroxides, hydroxycarbonates, borates, etc.; halogen containing flame retardants can be halogen flame retardant additives, reactive halogenated flame retardant monomers or polymers; phosphorous based flame retardants can be red phosphorous, inorganic phosphate, organic phosphorous based compounds, etc.; silicon based flame retardants can be silicon, silica compounds, etc.; nanometric particles can be nanoclay, carbon nanotube, nanoscale particulate additives, etc.

Ionic liquids may also be used as multifunctional additives. For example, an ionic liquid may be used as a lubricant and flame retardant, a plasticizer and flame retardant, a dispersant and flame retardant, and an antibacterial agent and flame retardant.

The proposed flame retardants can be used in many fields including plastics, textiles, paper, leather, wood, etc and can also be used as forest flame retardants.

### EXAMPLES

The materials and reagents used are either available from commercial suppliers or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In one embodiment, ionic liquids are modified to design biodegradable and nontoxic ionic liquids via incorporation of ethereal side chains. One such example is shown in FIG. 2. Greener Solvents; Room Temperature Ionic Liquids from Biorenewable Sources, Scott Handy, Chem. Eur. J. 2003, 9, 2938-2944

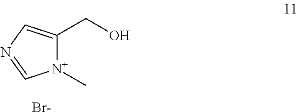

Hydroxymethyl imidazolium ionic liquid derivatives is synthesized from fructose according to the method reported by Totter and Handy in. Room Temperature Ionic Liquids: Different Classes and Physical Properties; Scott Handy; Current Organic Chemistry, 2005, 9, 959-988; Organic Letter, 2003, Vol. 5, No. 14, pp 2513-2515, Handy et al; Organic Syntheses, Coll. Vol. 3, p. 460 (1955); Vol. 24, p. 64 (1944), Totter et al

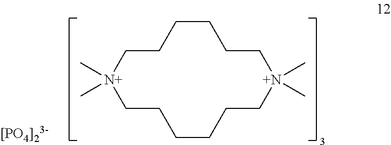

The cyclic diamidophosphate compound above is prepared according to chemistry described by Lall et al in Chem. Comm., 2000, 2413-2414

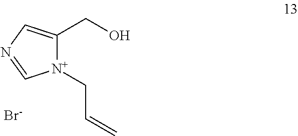

The allyl immadozolium bromide may be prepared according to chemistry described by Liu at al in Science of China, Series B: Chemistry, 2006, 149, 1, 385-401

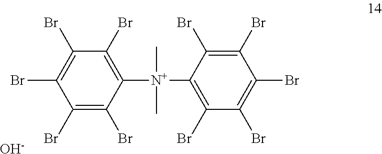

The brominated biphenylammonium compound above is prepared by methylation of the brominated biphenylamine described in Czech patent 233407 titled, “Preparation of brominated diphenyl amines as fire proofing agents”.

Compounding Treatment of Polyoxymethylene with 1-Butyl-3-methylimidazolium bromideand aluminum hydroxide:

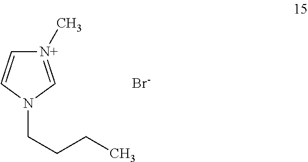

Aluminum hydroxide power (5 gms) is premixed with ionic liquid 15 (95 gms), then mixed with polyoxymethylene pellets (900 gms), and then melt-blended by a twin screw extruder at 170-185° C. with a screw rotation speed of 150-180 rpm. The extruded pellets are molded into standard bars for combustibility and mechanical performance tests through an injection-molding machine with a plasticizing temperature of 170-195° C.

Compounding treatment of polypropylene with intumescent flame retarding system using Triethylmethylphosphonium dibutyl phosphate >97.0% (CH)

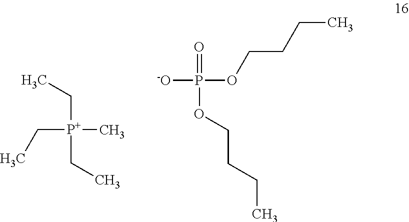

A mixture of ionic liquid 16 (2 gm), pentaerythritol (carbonization agent) (5 gm) and melamine (3 gms) are premixed and then mixed with polypropylene (90 gms). The mixture is then melt-blended by a twin screw extruder at 200° C. with a screw rotation speed of 150-180 rpm. The extruded pellets are molded into standard bars for combustibility and mechanical performance tests through an injection-molding machine with a plasticizing temperature of 230° C.

Treatment of PVC Using IL 15 with Antimony Trioxide:

A mixture of IL 15 (5 gm) and antimony trioxide (2 gm) are premixed, and then mixed with polyvinyl chloride resin (93 gm). The mixture is blended and molded into required shape and dimension in a similar manner as disclosed above.

### Treatment Of PVC Using IL 14 And Traditional Brominated Flame Retardant Tetrabromobisphenol A:

A mixture of IL 14 (3 gms), TBBPA (3 gm) are premixed, and mixed with PVC resin (94 gm). The mixture is blended and molded into required shape and dimension in a similar manner as disclosed above.

Treatment of High Density Polyethylene (HDPE) with Ionic Tributylmethylphosphonium Methyl Carbonate Liquid Modified Clay:

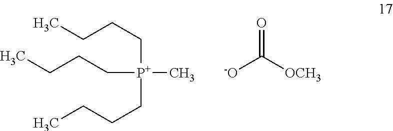

The surface of the clay is modified with ionic liquids through ion exchange reaction. HDPE (97 gm) and IL 17 modified clay (3 gm) are mixed, melt blended in ThermoHaake Rheomix with a screw speed of 60 rpm, and the mixing time for each sample is 15 min. The mixed samples are transferred to a mold and preheated at 180 C for 5 min and then pressed at 15 MPa followed by cooling them to room temperature while maintaining the pressure for 5 min.

Treatment of Polyimide 6 with Ionic Liquid/Carbon Nanotubes or Ionic Liquid/Carbon Nanofibers Using 1-Butylpyridinium Bromide

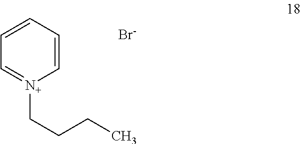

A mixture of IL 18 (3 gm) and carbon nanotubes or nanofibers (2 gm) are premixed, and then melt-blended and molded in a similar manner as disclosed above.

### Treatment of Polystyrene Via In-Situ Polymerization Method:

A mixture of styrene (95 gm), IL 15 (5 gm), AIBN (0.2 gm). The mixture is stirred magnetically under nitrogen at room temperature until a homogenous mixture is formed. The mixture is heated at 90° C. for pre-polymerization until a critical viscosity of the mixture is reached. The mixture was then transferred to an oven and kept isothermally at 60° C. for 24 h and then at 80° C. for 20 h. A copolymer containing IL N is obtained.

### Application IL Flame Retardants as a Components of Coating or Paint Layers:

Ionic liquid flame retardant 16 (5 gms) is mixed with 250 ml of paint and coating materials. The resulting material is used as a coating on flammable surfaces.

### Flame Retarding Finishing of Cotton Textile Materials:

A finishing aqueous solution containing 7% by weight IL flame retardant 11 is prepared. The cotton fleece is first immersed in the solution, then passed through a laboratory padder with two dips and two nips, dried at 90° C. for 3 min 45 s, and finally cured in a Mathis oven at 170° C. for 4 min.

### Flame Retarding Finishing of Leather Materials:

A finishing aqueous solution containing 7% by weight flame retardant 16 is prepared. And the finishing of leather can be done in a similar manner as used in textile finishing.

### Flame Retarding Treatment of Wood:

An aqueous impregnation solution is prepared containing 7% by weight IL 16. Test panels is prepared on A angustifolia. The impregnations are carried out at 201° C. in a vertical Pressure vessel of 251 capacity, provided with a vacuum pump and an air compressor. In all the cases, the vessel is loaded with the test panels to be impregnated; then the pressure is reduced by 400 mmHg for 30 min to remove air and vapor from the wood cells. The impregnants are added at the reduced pressure. Later on, the pressure is gradually increased until a final Value of 4781 mmHg (6.5 kgcm²) to facilitate the penetration; this stage lasts for 120 min. Next, creating light vacuum (approximately 50 mmHg for 10 min) to eliminate the excess of solution. Finally, the test panels are removed and rinsed with distilled water.

### Flame Treatment of Paper

An aqueous finishing solution containing 7% by weight IL 16 is prepared. The paper is treated by soaking the samples in the finishing solution for 10 min. The excess solution is removed by pressing the samples between two roll mills of a manually operated wringer.

### Wild Fire Protection:

50 weight % mixture of IL 12 is sprayed in wild forest for wild fire protection.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

What is claimed is:

1. A method of imparting a flame retarding property to a material comprising treating said material with an effective flame retarding amount of the composition of the formula: 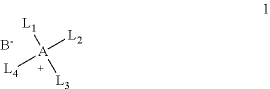
Wherein A is independently selected from a nitrogen, phosphorus or sulfur; when
A is nitrogen L₁, L₂, L₃ and L₄ are each independently selected from R₁, R₂, R₃ and R₄ and wherein R₁, R₂, R₃ and R₄ each independently form a single bond with N in a cyclic or acyclic structure; or, R₁, R₂ and R₃ combine to form an aromatic heterocycle further substituted by R₁, R₂, R₃ and R₄ bonded to N; R₁, R₂, R₃ and R₄ are each independently selected from the group consisting of hydrogen, alkyl, aryl, heterocyclyl, (C₁C₈)cycloalkyl, hetrocyclyl(C₁C₈)alkyl, aryl(C₁C₈)alkyl, heteroaryl and heteroaryl(C₁C₈)alkyl group that may be substituted or unsubstituted by halo, nitro, trifluoromethyl, trifluoromethoxy, methoxy, carboxy, NH₂, OH, SH, NHCH₃, N(CH₃)₂, SMe and cyano; R₁, R₂, R₃ and R₄ are not straight chain unsubstituted alkyl bonded to a quarterany N;
when A is sulfur Lund L₂ are R₁₂ and L₃ and L₄ are R₁₃, and R₁₄. R₁₂, R₁₃, and R₁₄ is selected from the group consisting of hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by halo, nitro, trifluoromethyl, trifluoromethoxy, methoxy, carboxy, NH₂, OH, SH, NHCH₃, N(CH₃)₂, SMe, cyano and the like;
when A is phosphorus, L₁, L₂, L₃ and L₄ are R₈, R₉, R₁₀, and R₁₁ wherein R₈, R₉, R₁₀, and R₁₁ are each independently selected from the group consisting of hydrogen, alkyl, aryl, heterocyclyl, (C₁C₈)cycloalkyl, hetrocyclyl(C₁C₈)alkyl, aryl(C₁C₈)alkyl, heteroaryl and heteroaryl(C₁C₈)alkyl group that may be substituted or unsubstituted by halo, nitro, trifluoromethyl, trifluoromethoxy, methoxy, carboxy, NH₂, OH, SH, NHCH₃, N(CH₃)₂, SMe, cyano and the like and wherein R₈, R₉, R₁₀, R₁₁ is not a hydroxymethyl group; when A is nitrogen and sulfur, B⁻ is X⁻ and X⁻ is selected from the group consisting of [PF₆]⁻, [NTf₂]⁻, [BR₁R₂R₃R₄]⁻, [BF₄]⁻, OH⁻, SCN⁻, SbF₆⁻, R₂PO₄⁻, RSO₃⁻, RSO₄, OTf⁻, tris(trifluoromethylsulfonyl)methide, [N(CN)₂]⁻, [CH₃CO₂]⁻, [CF₃CO₂]⁻, [NO₃]⁻, Br⁻, Cl⁻, I⁻, [Al₂Cl₇]⁻, [AlCl₄]⁻, oxalate, dicarboxylates and tricarboxylate, formate, phosphate and aluminate or a suitably substituted negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted;
when A is phosphorus B⁻ is X₁⁻ and X₁⁻ selected from the group consisting of [PF₆]⁻, [NTf₂]⁻, [BR₁R₂R₃R₄]⁻, [BF₄]⁻, OH⁻, SCN⁻, SBF₆⁻, R₂PO₄⁻, RSO₃⁻, RSO₄, OTf⁻, tris(trifluoromethylsulfonyl)methide [N(CN)₂]⁻, [CH₃CO₂]⁻, [CF₃CO₂]⁻, [NO₃]⁻, [Al2Cl₇]⁻, [AlCl₄]⁻, oxalate, dicarboxylates and tricarboxylate, formate, phosphate, I⁻ and aluminate and the like or a suitably substituted negatively charged functional group on an alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted and wherein X₁⁻ is not a Br⁻ and Cl⁻; when one of the four R₈, R₉, R₁₀, R₁₁ group is a C₁ to C₁₈ (CH₂)ₙ chain bonded to P., X₁⁻ is not SBF6, PF6, BF4, AlF6, triflate, AsF6, (B[C6F5]4⁻), (B[C6H3(C6H3(CF3)2]4⁻), tetra phenyl borate, hexafluorotitanate, pentachlorotitanate, pentachlorostannate, hexafluorogermanate, hexafluorosilicate, hexafluoronickelate, or hexafluorozirconate

2. The method according to claim 1 of imparting a flame retarding property to a material comprising treating said material with an effective flame retarding amount of the composition of the formula:

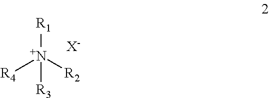

3. The method according to claim 1 of formula:

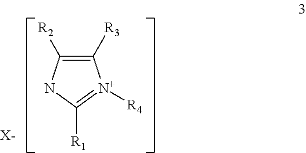

4. The method according to claim 1 of formula: 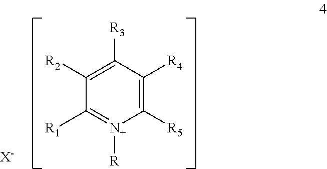
Wherein R₅ and R are an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like;
R₅ R₆, and R₇ may be a reactive group that serves to bond the ionic liquid into a polymer such as a vinyl, epoxide, acrylate, isocyanate, acyl halide

5. The method according to claim 1 of formula: 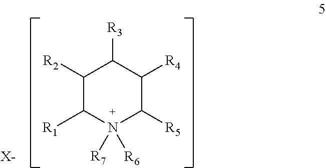
Wherein R₅ R₆, and R₇ are an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like;
R₅ R₆, and R₇ may be a reactive group that serves to bond the ionic liquid into a polymer such as a vinyl, epoxide, acrylate, isocyanate, acyl halide

6. The method according to claim 1 of formula: 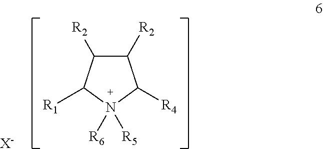
Wherein R₅ and R₆ are an organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like;
R₅ R₆, and R₇ may be a reactive group that serves to bond the ionic liquid into a polymer such as a vinyl, epoxide, acrylate, isocyanate, acyl halide

7. The method according to claim 1 of formula: 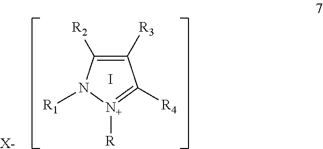
Wherein R is organic group which maybe a hydrogen, alkyl, aryl, heterocyclyl, (C₁C₈)cycloalkyl, hetrocyclyl(C₁C₈)alkyl, aryl(C₁C₈)alkyl, heteroaryl or heteroaryl(C₁C₈)alkyl group that may be substituted or unsubstituted by a functional group like halo, nitro, trifluoromethyl, trifluoromethoxy, methoxy, carboxym, NH₂, OH, SH, NHCH₃, N(CH₃)₂, SMe, cyano and the like;
R₅ R₆, and R₇ may be a reactive group that serves to bond the ionic liquid into a polymer such as a vinyl, epoxide, acrylate, isocyanate, acyl halide

8. The method according to claim 1 of formula:

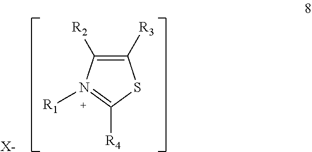

9. The method according to claim 1 of imparting a flame retarding property to a material comprising treating said material with an effective flame retarding amount of the composition of the formula:

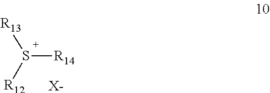

10. The method according to claim 1 of imparting a flame retarding property to a material comprising treating said material with an effective flame retarding amount of the composition of the formula:

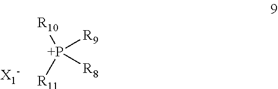

11. The method according to claim 1 of imparting a flame retarding property to a material comprising treating said material with an effective flame retarding amount of the composition of claim 1 in combination with another ionic liquid compound.

12. The method according to claim 1 of imparting a flame retarding property to a material comprising treating said material with an effective flame retarding amount of the composition of claim 1 in combination with a mineral flame retardant.

13. The method according to claim 1 of imparting a flame retarding property to a material comprising treating said material with an effective flame retarding amount of the composition claim 1 combined with a halogenated flame retardant.

15. The method according to claim 1 of imparting a flame retarding property to a material comprising treating said material with an effective flame retarding amount of the composition of claim 1 combined with a phosphorus based flame retardant.

17. The method according to claim 1 of imparting a flame retarding property to a material comprising treating said material with an effective flame retarding amount of the composition of claim 1 combined with a nitrogen based flame retardant,

18. The method according to claim 1 of imparting a flame retarding property to a material comprising treating said material with an effective flame retarding amount of the composition of claim 1 combined with silicon based flame retardants.

19. The method according to claim 1 of imparting a flame retarding property to a material comprising treating said material with an effective flame retarding amount of the composition of claim 1 combined with nanometric particles.

20. The method according to claim 1 of imparting a flame retarding property to a textile material comprising treating said textile with an effective flame retarding amount of the composition of claim

21. The method according to claim 1 of imparting a flame retarding property to a plastic material comprising treating said plastic material with an effective flame retarding amount of the composition of claim 1.

22. The method according to claim 1 of imparting a flame retarding property to a leather comprising treating said leather with an effective flame retarding amount of the composition of claim 1.

23. The method according to claim 1 of imparting a flame retarding property to paper comprising treating said paper with an effective flame retarding amount of the composition of claim 1.

24. The method according to claim 1 of imparting a flame retarding property to wood comprising treating said wood with an effective flame retarding amount of the composition of claim 1.

25. The method according to claim 1 of imparting a flame retarding property to a rubber comprising treating said rubber with an effective flame retarding amount of the composition of claim 1.

26. The method according to claim 1 of using an effective flame retarding amount of the composition of claim 1 as a wild fire retardant.

27. A composition comprising an effective flame retarding amount of the composition of claim 1 in combination with a material selected from the group consisting of plastic, textile, wood, leather, paper and rubber.

28. The plastic composition of claim 27 comprising an effective flame retarding amount of the composition of claim 1 as a flame retardant.

29. The textile composition of claim 27 comprising an effective flame retarding amount of the composition of claim 1 as a flame retardant.

30. The wood composition of claim 27 comprising an effective flame retarding amount of the composition of claim 1 as a flame retardant.

31. The paper composition of claim 27 comprising an effective flame retarding amount of the composition of claim 1 as a flame retardant.

32. The leather composition of claim 27 comprising an effective flame retarding amount of the composition of claim 1 as a flame retardant.

33. The rubber composition of claim 27 comprising an effective flame retarding amount of the composition of claim 1 as a flame retardant.

34. A method of imparting a flame retarding property to a material comprising treating said material with an effective flame retarding amount of formula A⁻B⁺ wherein the cationionic or the anionic species is an ionic liquid ion and its counter ion is an ion bonded to a polymer.
